(19) United States
(12) Patent Application Publication
(10) Pub. No.: US 2020/0297591 A1
(43) Pub. Date: Sep. 24, 2020

(54) Topical Phospholipid Formulations and Methods for Preparing the Same

(71) Applicant: Essential Lipid Technologies, LLC, Winchester, MA (US)

(72) Inventors: Thomas GLONEK, Oak Park, IL (US); Jack GREINER, Winchester, MA (US); Donald KORB, Boston, MA (US); Hridaya BHARGAVA, Sharron, MA (US)

(21) Appl. No.: 16/816,362

(22) Filed: Mar. 12, 2020

Related U.S. Application Data
(62) Division of application No. 16/356,393, filed on Mar. 18, 2019.
(60) Provisional application No. 62/644,849, filed on Mar. 19, 2018.

(51) Int. Cl. A61K 8/06; A61K 8/55; A61K 8/92; A61K 8/36; A61K 8/67

(52) U.S. Cl. CPC A61K 8/06; A61K 8/553; A61K 8/927; A61Q 17/00; A61K 8/922; A61K 8/678; A61K 8/36

(57) ABSTRACT

A topical phospholipid formulation is provided that includes a phospholipid component. The phospholipid component is anionic and exhibits a net negative charge at physiologic pH. Methods of preparing such a formulation are also provided.

### CROSS-REFERENCE TO RELATED APPLICATIONS

The present application is a divisional of U.S. Ser. No. 16/356,393 filed Mar. 18, 2019 which claims priority to U.S. Provisional Application Ser. No. 62/644,849 filed Mar. 19, 2018.

### FIELD OF THE DISCLOSURE

A topical phospholipid formulation is provided that includes at least one phospholipid component. Methods of preparing such a formulation are also provided.

### BACKGROUND

The principal function of the skin is to provide a barrier against excessive water evaporation, microorganisms, and physical and chemical trauma. The epithelial cell membranes of the skin are comprised of large amounts of lipids, phospholipids, and water that form lipid bilayers. Disruption of these bilayers compromises both the barrier function and the hydration status of the skin potentially resulting in a range of disorders including, but not limited to, drying, flaking, cracking, accelerated aging, inflammation, infection, impaired wound healing, and discomfort. In the case of the skin epidermis, the lipids of the more superficial layers, especially the stratum corneum, form polar membrane bilayers or lamellae. The inherent physicochemical properties of these polar lipids are integral to the formation of these extracellular lipid bilayers that are the building blocks of the skin's lamellae system.

Available treatments for addressing compromised skin are limited in many ways. Some formulations merely attempt to treat the symptoms, e.g. dryness, at a superficial level rather than the underlying cause of functionally-compromised lipid membranes. Other formulations do not satisfactorily penetrate the skin and are thus unable to deliver potentially therapeutic agents to repair and/or replenish damaged and/or defective lipid membranes or lamellae. As such, there remains an unmet need for topical formulations and methods of preparing such topical formulations that effectively restores the biochemical, physical, and functional integrity of the lipid membranes and lamellae that are critical to the fundamental nature of the skin and, by extension, their role as a viable and responsive barrier to external and internal assaults.

### SUMMARY

According to one aspect, a method of preparing a topical phospholipid formulation is provided. The method includes the steps of preparing an aqueous phase; preparing an oil phase; mixing the oil phase and aqueous phase to form an emulsion; introducing at least one mixture comprising a phospholipid component to the emulsion to form an emulsion composition; adjusting the pH of the emulsion composition to physiologic pH; and homogenizing the emulsion composition to form the topical phospholipid formulation. The phospholipid component includes an anionic mole fraction of between from about 20 mole percent to about 100 mole percent of one or more net anionic phospholipid molecules, with the remaining balance of the phospholipid component including one or more neutral zwitterionic phospholipids at the physiologic pH. According to one embodiment, the physiologic pH is from about 9.0 to about 5.0. According to one embodiment, the topical phospholipid formulation is a cream. According to one embodiment, the step of preparing the aqueous phase includes the steps of: heating water; and adding one or more of an emulsifier, emulsion stabilizer, and rheology modifier to the water. According to one embodiment, the step of preparing an oil phase includes mixing at least two or more components selected from the group consisting of cetyl alcohol, beeswax, isopropyl palmitate, oleic acid, sorbitan stearate, stearyl alcohol, stearic acid, glyceryl stearate, food shortening, antioxidant, vitamin E acetate, ascorbyl palmitate, and an ester. According to one embodiment, the step of mixing the oil phase and aqueous phase to form an emulsion includes mixing for about 10 minutes to about 20 minutes at a temperature of from about 70° C. to about 80° C. According to one embodiment, the step of adjusting the pH is carried out by introducing 0.1N sodium hydroxide or citric acid to the emulsion. According to one embodiment, the step of homogenizing the emulsion composition is carried out at from about 20° C. to about 35° C. According to one embodiment, the method further includes the step of introducing a 10% w/v sodium hydroxide solution to the emulsion. According to one embodiment, the method further includes the step of introducing one or more preservative/fragrance solution to the emulsion composition. According to one embodiment, the method further includes the step of combining the phospholipid component with one or more preservative and, optionally, one or more carrier, to form the mixture.

According to one aspect, a topical phospholipid formulation is provided that is prepared according to the steps provided herein.

According to another aspect, a topical phospholipid formulation is provided that includes a phospholipid component. The phospholipid component includes an anionic mole fraction of between from about 20 mole percent to about 100 mole percent of one or more net anionic phospholipid molecules, with the remaining balance of the phospholipid component including one or more neutral zwitterionic phospholipids at physiologic pH. According to one embodiment, the physiologic pH is from about 9.0 to about 5.0. According to one embodiment, topical phospholipid formulation further includes one or more of a viscosity modifier, emulsifier, antioxidant, vitamin, solvent, fragrance, preservative, base, acid, carrier, or any combination thereof. According to one embodiment, the carrier is water, an alcohol, or a combination thereof. According to one embodiment, the phospholipid formulation is a lotion, hydrogel, oil, emulsion, paste, polish, or cream. According to one embodiment, the phospholipid component is present in a concentration of from about 0.1% w/w to about 10% w/w based on the total weight of the formulation. According to one embodiment, the phospholipid component includes one or more phospholipid molecules selected from the group consisting of lysophosphatidic acid, lysodiphosphatidylglycerol, phosphatidylglycerol, lysophosphatidylethanolamine, phosphatidic acid, diphosphatidylglycerol, ethanolamine plasmalogen, phosphatidylethanolamine, phosphatidylserine, dimethylphosphatidylethanolamine, lysophosphatidylcholine, phosphatidylinositol, and phosphatidylcholine. According to one embodiment, the phospholipid component includes an anionic mole fraction of between from about 20 mole percent to about 40 mole percent of one or more net anionic phospholipid molecules.

### DETAILED DESCRIPTION

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein the term “phospholipid” may refer to any variety of lipids containing a phosphate group. Particular embodiments include glycerol derivatives in which one of its hydroxyl groups is esterified with phosphoric acid and the other two hydroxyls are esterified with long-chain fatty acids, which may be equal or different from each other. A saturated phospholipid will be that whose fatty acids only have single (not multiple) carbon-carbon links.

A charged or “polar” phospholipid is a molecule containing a phosphate group and also one or more negative or positive charges.

As used herein, the term “anionic” is meant to refer to the state of being net negatively charged at physiologic pH.

As used herein, the term “carrier” refers to a composition or a diluent that does not cause significant irritation to the skin and does not abrogate the biological activity and properties of the phospholipid component.

As used herein, the term “topical formulation” refers to a cream, gel, salve, ointment, or similar formulation particularly suited, for example, for application to the skin.

As used herein, the term “zwitterionic” refers to a molecule with functional groups that include at least one positive and at least one negative electrical charge, the net charge of which is zero.

As used herein, the term “homogenizing” or “homogenize” is used to describe a step of forming a stable dispersion of an oil phase in a water/aqueous phase (i.e., the step of forming the topical formulation). A mechanical mixing effect may be employed to reduce oil phase particle size and produce a more uniform particle size. Covalent bonds are maintained but hydrophobic-hydrophilic interactions are altered.

The present disclosure provides topical phospholipid formulations that include a phospholipid component. The anionic mole fraction of the total phospholipid component is typically between from about 20 mole percent to about 100 mole percent of one or more net anionic phospholipid molecules, with the remaining balance of the phospholipid component including one or more neutral zwitterionic phospholipids at physiologic pH values. The present disclosure also provides methods of preparing topical formulations that include a phospholipid component.

According to one embodiment, the topical formulations as provided herein seek to treat injured cells that are leaking because their membranes have been disrupted or injured. The present topical formulations treat the flaws and repair the cells including the tissue. The topical formulations include a phospholipid component and overcomes the deficiencies of existing topical formulations by the unique engineering of phospholipid bilayer components, where an anionic polar head group interacts with water and the hydrocarbon tails interact with oil. The two interconnected units of each molecule thus act like a “zipper” to hold together the dissimilar oil and water, repairing the separated lipid bilayers of the lamellae system into a functional film and thus creating an environment conducive to the repair and restoration of compromised or damaged skin tissue. The phospholipid component includes one or more phospholipids, the aggregate of which possesses a net negative charge at physiologic pH and at the final pH of the product preparation. As a result, the topical formulations as provided herein exhibit therapeutic properties.

According to one embodiment, the topical formulations as provided herein include a phospholipid component. According to one embodiment, the phospholipid component includes an anionic mole fraction of between from about 20 mole percent to about 100 mole percent of one or more net anionic phospholipid molecules, with the remaining balance of the phospholipid component including one or more neutral zwitterionic phospholipids at physiologic pH. According to another embodiment, the phospholipid component includes an anionic mole fraction of between from about 20 mole percent to about 40 mole percent of one or more net anionic phospholipid molecules, with the remaining balance of the phospholipid component including one or more neutral zwitterionic phospholipids at physiologic pH. According to yet another embodiment, the phospholipid component includes an anionic mole fraction of between from about 25 mole percent to about 35 mole percent of one or more net anionic phospholipid molecules, with the remaining balance of the phospholipid component including one or more neutral zwitterionic phospholipids at physiologic pH.

According to one embodiment, the phospholipid component is present in the final formulation at a concentration suitable for the purpose or end use of the formulation. Conditions and disorders that present more severe symptoms may require a higher concentration of the phospholipid component. According to one embodiment, the phospholipid component is present in the topical formulations at a concentration of typically from about 0.01% w/w to about 10% w/w based on the total weight of the final formulation. According to one embodiment, the phospholipid component is present in the topical formulations at a concentration of typically from about 0.1% w/w to about 5% w/w. According to one embodiment, the phospholipid component is present in the topical formulations at a concentration of typically about 0.1% w/w. According to one embodiment, the phospholipid component is present in the topical formulations at a concentration of typically about 1% w/w. According to one embodiment, the phospholipid component is present in the topical formulations at a concentration of typically about 2% w/w. According to one embodiment, the phospholipid component is present in the topical formulations at a concentration of typically about 3% w/w. According to one embodiment, the phospholipid component is present in the formulations at a concentration of typically about 4% w/w. According to one embodiment, the phospholipid component is present in the formulations at a concentration of typically about 5% w/w.

According to one embodiment, the phospholipid component includes one or more phospholipids. Suitable phospholipids include, but are not limited to, one or more of lysophosphatidic acid, lysodiphosphatidylglycerol, phosphatidylglycerol, lysophosphatidylethanolamine, phosphatidic acid, diphosphatidylglycerol, ethanolamine plasmalogen, phosphatidylethanolamine, phosphatidylserine, dimethylphosphatidylethanolamine, lysophosphatidylcholine, phosphatidylinositol, phosphatidylcholine, or any combination thereof.

According to one embodiment, the phospholipid component itself is composed of or typically includes from about 0.1% w/w to about 100% w/w lysophosphatidic acid based on the total weight of the phospholipid component. According to one embodiment, the phospholipid component itself is composed of or typically includes from about 0.1% w/w to about 10% w/w. According to one embodiment, the phospholipid component itself is composed of or typically includes from about 0.5% w/w to about 1.5% w/w lysophosphatidic acid. According to one embodiment, the phospholipid component itself is composed of or typically includes from about 0.8% w/w to about 1.0% w/w lysophosphatidic acid.

According to one embodiment, the phospholipid component itself is composed of or typically includes from about 0.1% w/w to about 100% w/w lysodiphosphatidylglycerol based on the total weight of the phospholipid component. According to one embodiment, the phospholipid component itself is composed of or typically includes from about 0.1% w/w to about 10% w/w lysodiphosphatidylglycerol. According to one embodiment, the phospholipid component itself is composed of or typically includes from about 0.1% w/w to about 1.0% w/w lysodiphosphatidylglycerol. According to one embodiment, the phospholipid component itself is composed of or typically includes from about 0.5% w/w to about 0.7% w/w lysodiphosphatidylglycerol.

According to one embodiment, the phospholipid component itself is composed of or typically includes from about 0.1% w/w to about 100% w/w phosphatidylglycerol based on the total weight of the phospholipid component. According to one embodiment, the phospholipid component itself is composed of or typically includes from about 0.1% w/w to about 10% w/w phosphatidylglycerol. According to one embodiment, the phospholipid component itself is composed of or typically includes from about 1.0% w/w to about 5.0% w/w phosphatidylglycerol. According to one embodiment, the phospholipid component itself is composed of or typically includes from about 2.0% w/w to about 3.0% w/w phosphatidylglycerol.

According to one embodiment, the phospholipid component itself is composed of or typically includes from about 0.1% w/w to about 100% w/w lysophosphatidylethanolamine based on the total weight of the phospholipid component. According to one embodiment, the phospholipid component itself is composed of or typically includes from about 0.1% w/w to about 10% w/w lysophosphatidylethanolamine. According to one embodiment, the phospholipid component itself is composed of or typically includes from about 0.5% w/w to about 3.0% w/w lysophosphatidylethanolamine. According to one embodiment, the phospholipid component itself is composed of or typically includes from about 1.0% w/w to about 2.0% w/w lysophosphatidylethanolamine.

According to one embodiment, the phospholipid component itself is composed of or typically includes from about 0.1% w/w to about 100% w/w phosphatidic acid based on the total weight of the phospholipid component. According to one embodiment, the phospholipid component itself is composed of or typically includes from about 0.1% w/w to about 40% w/w phosphatidic acid. According to one embodiment, the phospholipid component itself is composed of or typically includes from about 10% w/w to about 30% w/w phosphatidic acid. According to one embodiment, the phospholipid component itself is composed of or typically includes from about 15% w/w to about 20% w/w phosphatidic acid.

According to one embodiment, the phospholipid component itself is composed of or typically includes from about 0.1% w/w to about 100% w/w diphosphatidylglycerol based on the total weight of the phospholipid component. According to one embodiment, the phospholipid component itself is composed of or typically includes from about 0.1% w/w to about 10% w/w diphosphatidylglycerol. According to one embodiment, the phospholipid component itself is composed of or typically includes from about 0.5% w/w to about 3.0% w/w diphosphatidylglycerol. According to one embodiment, the phospholipid component itself is composed of or typically includes from about 1.0% w/w to about 2.0% w/w diphosphatidylglycerol.

According to one embodiment, the phospholipid component itself is composed of or typically includes from about 0.1% w/w to about 100% w/w ethanolamine plasmalogen based on the total weight of the phospholipid component. According to one embodiment, the phospholipid component itself is composed of or typically includes from about 0.1% w/w to about 10% w/w ethanolamine plasmalogen. According to one embodiment, the phospholipid component itself is composed of or typically includes from about 1.0% w/w to about 7.0% w/w ethanolamine plasmalogen. According to one embodiment, the phospholipid component itself is composed of or typically includes from about 3.0% w/w to about 4.0% w/w ethanolamine plasmalogen.

According to one embodiment, the phospholipid component itself is composed of or typically includes from about 0.1% w/w to about 100% w/w phosphatidylethanolamine based on the total weight of the phospholipid component. According to one embodiment, the phospholipid component itself is composed of or typically includes from about 0.1% w/w to about 40% w/w phosphatidylethanolamine. According to one embodiment, the phospholipid component itself is composed of or typically includes from about 10% w/w to about 35% w/w phosphatidylethanolamine. According to one embodiment, the phospholipid component itself is composed of or typically includes from about 25% w/w to about 30% w/w phosphatidylethanolamine.

According to one embodiment, the phospholipid component itself is composed of or typically includes from about 0.1% w/w to about 100% w/w phosphatidylserine based on the total weight of the phospholipid component. According to one embodiment, the phospholipid component itself is composed of or typically includes from about 0.1% w/w to about 10% w/w phosphatidylserine. According to one embodiment, the phospholipid component itself is composed of or typically includes from about 0.5% w/w to about 3.0% w/w phosphatidylserine. According to one embodiment, the phospholipid component itself is composed of or typically includes from about 1.0% w/w to about 2.0% w/w phosphatidylserine.

According to one embodiment, the phospholipid component itself is composed of or typically includes from about 0.1% w/w to about 100% w/w dimethylphosphatidylethanolamine based on the total weight of the phospholipid component. According to one embodiment, the phospholipid component itself is composed of or typically includes from about 0.1% w/w to about 10% w/w dimethylphosphatidylethanolamine. According to one embodiment, the phospholipid component itself is composed of or typically includes from about 0.5% w/w to about 3.0% w/w dimethylphosphatidylethanolamine. According to one embodiment, the phospholipid component itself is composed of or typically includes from about 1.0% w/w to about 2.0% w/w dimethylphosphatidylethanolamine.

According to one embodiment, the phospholipid component itself is composed of or typically includes from about 0.1% w/w to about 100% w/w lysophosphatidylcholine based on the total weight of the phospholipid component. According to one embodiment, the phospholipid component itself is composed of or typically includes from about 0.1% w/w to about 10% w/w lysophosphatidylcholine. According to one embodiment, the phospholipid component itself is composed of or typically includes from about 0.5% w/w to about 3.0% w/w lysophosphatidylcholine. According to one embodiment, the phospholipid component itself is composed of or typically includes from about 1.0% w/w to about 2.0% w/w lysophosphatidylcholine.

According to one embodiment, the phospholipid component itself is composed of or typically includes from about 0.1% w/w to about 100% w/w phosphatidylinositol based on the total weight of the phospholipid component. According to one embodiment, the phospholipid component itself is composed of or typically includes from about 0.1% w/w to about 40% w/w phosphatidylinositol. According to one embodiment, the phospholipid component itself is composed of or typically includes from about 10% w/w to about 30% w/w phosphatidylinositol. According to one embodiment, the phospholipid component itself is composed of or typically includes from about 20% w/w to about 25% w/w phosphatidylinositol.

According to one embodiment, the phospholipid component itself is composed of or typically includes from about 0.1% w/w to about 100% w/w phosphatidylcholine based on the total weight of the phospholipid component. According to one embodiment, the phospholipid component itself is composed of or typically includes from about 0.1% w/w to about 10% w/w phosphatidylcholine. According to one embodiment, the phospholipid component itself is composed of or typically includes from about 10% w/w to about 25% w/w phosphatidylcholine. According to one embodiment, the phospholipid component itself is composed of or typically includes from about 15% w/w to about 20% w/w phosphatidylcholine.

According to a particular embodiment, the phospholipid component is composed or typically includes from about 0.1% w/w to about 10% w/w lysophosphatidic acid; from about 0.1% w/w to about 10% w/w lysodiphosphatidylglycerol; from about 0.1% w/w to about 10% w/w phosphatidylglycerol; from about 0.1% w/w to about 10% w/w lysophosphatidylethanolamine; from about 0.1% w/w to about 40% w/w phosphatidic acid; from about 0.1% w/w to about 10% w/w diphosphatidylglycerol; from about 0.1% w/w to about 10% w/w ethanolamine plasmalogen; from about 0.1% w/w to about 40% w/w phosphatidylethanolamine; from about 0.1% w/w to about 10% w/w phosphatidylserine; from about 0.1% w/w to about 10% w/w dimethylphosphatidylethanolamine; from about 0.1% w/w to about 10% w/w lysophosphatidylcholine; from about 0.1% w/w to about 40% w/w phosphatidylinositol; and from about 0.1% w/w to about 10% w/w phosphatidylcholine.

According to one embodiment, the topical formulations as provided herein may also include one or more thickening agent, emulsion stabilizer, rheology modifier, or viscosity modifier. According to one embodiment, the one or more thickening agent, emulsion stabilizer, rheology modifier, or viscosity modifier may be present in an amount of from about 0.1% w/w to about 10% w/w based on the total weight of the final formulation. According to one embodiment, the one or more thickening agent, emulsion stabilizer, rheology modifier, or viscosity modifier may be present in an amount of from about 1.0% w/w to about 5% w/w. Such thickening agents, emulsion stabilizers, rheology modifiers, or viscosity modifiers include, but are not limited to, cross-linked polyacrylic acid polymer, mineral oil, light mineral oil, natural oil (e.g., vegetable, corn, canola, sunflower, soybean, olive, coconut, cocoa, peanut, almond, cottonseed, persic, sesame, squalane, castor, cod liver, etc.) hydrogenated vegetable oil, partially hydrogenated oil, beeswax, polyethoxylated beeswax, paraffin, normal wax, medium chain monoglycerides, diglycerides and triglycerides, higher aliphatic alcohols, higher aliphatic acids, long chain fatty acids, saturated or unsaturated fatty acids, hydrogenated fatty acids, fatty acid glycerides, polyoxyethylated oleic glycerides, monoglycerides and diglycerides, mono-, bi- or tri-substituted glycerides, glycerol mono-oleate esters, glycerol mono-caprate, glyceryl monocaprylate, propylene glycol, propylene glycol dicaprylate, propylene glycol monolaurate, glyceryl palmitostearate, glyceryl behenate, diethyleneglycol palmitostearate, polyethyleneglycol stearate, polyoxyethyleneglycol palmitostearate, glyceryl mono palmitostearate, cetyl palmitate, polyethyleneglycol palmitostearate, dimethylpolysiloxane, mono- or di-glyceryl behenate, fatty alcohols associated with polyethoxylate fatty alcohols, cetyl alcohol, octyl dodecanol, myristyl alcohol, myristyl myristate, myristyl laurate, isopropyl myristate, isopropyl palmitate, stearic acid, stearyl alcohol or any combination thereof.

According to one embodiment, the topical formulations as provided herein may also include one or more emulsifiers. According to one embodiment, the one or more emulsifiers may be present in an amount of from about 0.1% w/w to about 5% w/w based on the total weight of the final formulation. According to one embodiment, the one or more emulsifiers may be present in an amount of from about 1.0% w/w to about 3.0% w/w. Suitable emulsifiers include sorbitan stearate, acacia, cholesterol, diethanolamine, glyceryl monostearate, glyceryl, stearate, lanolin alcohols, lecithin, mono- and di-glycerides, monoethanolamine, oleic acid, oleyl alcohol poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate, stearic acid, trolamine, polyethylene glycol sorbitan monostearate, emulsifying wax or any combination thereof.

According to one embodiment, the topical formulations as provided herein may also include one or more antioxidants. According to one embodiment, the one or more antioxidants is present in an amount of from about 0.01% w/w to about 10% w/w based on the total weight of the final formulation. According to one embodiment, the one or more antioxidants is present in an amount of from about 0.02% w/w to about 5.0% w/w. Suitable antioxidants include, but are not limited to, tocopherols (e.g., alpha tocopherol, beta tocopherol, gamma tocopherol, or delta tocopherol), butylated hydroxytoluene (BHT), butylated hydroxyanisole (BHA), lecithin, citric acid, ascorbic acid, phenolic diterpenes (e.g., carnosic acid, carnosol, rosmanol, epirosmanol, isorosmanol, or methyl carnosate), rosmarinic acid, eugenol, eugenyl acetate, clove bud extract, methanolic extract, tea catechins (e.g., epigallocatechin gallate, epicatechin gallate, epigallocatechin, or epicatechin) or any combination thereof.

According to one embodiment, the topical formulations as provided herein may also include one or more additives. According to one embodiment, the one or more additives is present in an amount of from about 1.0% w/w to about 10% w/w based on the total weight of the final formulation. According to one embodiment, the one or more additives is present in an amount of from about 2.0% w/w to about 7.5% w/w. Suitable additives include, but are not limited to, non-ionic, anionic or cationic polymers, preservatives, oils, pH regulators, waxes, fragrances, anti-fatting agents, sequestrating agents, perfumes, dyes, cationic surfactants, proteins, silicones, surfactants, organic solvents and any other additive conventionally used in the topical formulation field. Other suitable additional additives include, but are not limited to, solid emollients, surface active agents, gum, humectant, thickener, powder diluent, dispersant, or carrier so as to facilitate the distribution of the topical formulation when applied.

According to one embodiment, the topical formulations as provided herein may also include one or more solvents. According to one embodiment, the one or more solvents is present in an amount of from about 0.5% w/w to about 10% w/w based on the total weight of the final formulation. According to one embodiment, the one or more solvents is present in an amount of from about 2.0% w/w to about 7.5% w/w. Suitable solvents include, but are not limited to, low alcohols, for example ethanol or isopropanol, glycerol, glycols or glycol ethers such as the monobutyl ether of ethyleneglycol, propyleneglycol, or the monoethylether or the monomethylether of diethyleneglycol or any combination thereof. The solvents are both cosmetically acceptable as well as therapeutically acceptable for topical formulations.

According to one embodiment, the topical formulations as provided herein may also include one or more therapeutically acceptable carriers. According to one embodiment, the one or more carriers may be present in an amount of from about 30% w/w to about 95% w/w based on the total weight of the final formulation. According to one embodiment, the one or more carriers may be present in an amount of from about 40% w/w to about 85% w/w. The carriers as provided herein do not abrogate the biological activity and properties of the applied active agent. According to a particular embodiment, the carrier penetrates the epithelial layer of the skin and may penetrate the stratum corneum to provide lubrication and moisture to the skin.

According to one embodiment, topical carriers typically include other agents and ingredients commonly employed in dermatological and cosmetic products, and preparations such as salves, creams, ointments and lotions. Specific carriers include, but are not limited to, water, liquid alcohols, liquid glycols, liquid polyalkylene glycols, liquid esters, liquid amides, liquid protein hydrosylates, liquid alkylated protein hydrosylates, liquid lanolin and lanolin derivatives, and other like materials. Other exemplary carriers include, but are not limited to, water, alcohols inclusive of both monohydric and polyhydric alcohols (e.g. ethanol, isopropanol, glycerol, sorbitol, 2-methoxyethanol, diethylene glycol, ethylene glycol, hexylene glycol, mannitol, cetyl alcohol and propylene glycol), ethers (e.g., diethyl or dipropyl ether), polyethylene glycols, methoxypolyoxyethylenes, carbowaxes having molecular weights ranging from 200 to 20,000, polyoxyethylene glycerols, polyoxyethylene, sorbitols, stearoyl diacetin, or any combination thereof. According to one embodiment, a suitable carrier includes both alcohol and water.

According to one embodiment, the topical formulations as provided herein may optionally include one or more vitamins. According to one embodiment, the one or more vitamins may be present in an amount of from about 0.01% w/w to about 5.0% w/w based on the total weight of the final formulation. According to one embodiment, the one or more vitamins may be present in an amount of from about 0.02% w/w to about 2.5% w/w. Suitable vitamins include, but are not limited to, ascorbic acid, citric acid, vitamin D, vitamin E, vitamin K, niacin, pantothenic acid, choline, inositol, folic acid or any combination thereof.

According to one embodiment, the topical formulations as provided herein may optionally include one or more nutraceuticals. According to one embodiment, the one or more nutraceuticals may be present in an amount of from about 0.01% w/w to about 5.0% w/w based on the total weight of the final formulation. According to one embodiment, the one or more nutraceuticals may be present in an amount of from about 0.02% w/w to about 2.5% w/w. Suitable nutraceuticals include, but are not limited to, amino acids, terpenoids (e.g., carotenoid terpenoids and non-carotenoid terpenoids), herbal supplements, homeopathic supplements, glandular supplements, polyphenolics, flavonoid polyphenolics, phenolic acids, curcumin, resveratrol, lignans, glucosinolates, isothiocyanates, indoles, thiosulfinates, phytosterols, anthraquinones, capsaicin, piperine, chlorophyll, betaine, oxalic acid, acetyl-L-carnitine, allantoin, androstenediol, androstendione, betaine (trimethylglycine), caffeine, calcium pyruvate (pyruvic acid), carnitine, carnosine, carotene, carotenoid, choline, chlorogenic acid, cholic acid, chondroitin sulfate, chondroitin sulfate, cholestan, chrysin, coenzyme Q10, conjugated linoleic acid, corosolic acid, creatine, dehydroepiandrosterone, dichlorophen, diindolymethane, dimethylglycine, dimercapto succinic acid, ebselen, ellagic acid, enzymes, fisetin, formononetin, glucaric acid (glucarate), glucosamine (HCl or sulfate), glucosamine (N-acetyl), glutathione, hesperidine, hydroxy-3-methylbutyric acid, 5-hydroxytryptophan, indole-3-carbinol, inositol, isothiocyanates, linolenic acid-gamma, lipoic acid (alpha), melatonin, methylsulfonylmethane, minerals, naringin, pancreatin, para-aminobenzoic acid, paraben (methyl or propyl), phenolics, phosphatidylcholine, phosphatidylserine, phospholipids, phytosterols, progesterone, pregnenolone, omega-3 fatty acids, fatty acids, quercetin, resveratrol, D-ribose, rutin, S-adenosylmethionine, salicylic acid, sulforaphane, tartaric acid, taxifolin, tetrahydropalmatine, theophyline, theobromine, tigogenin, troxerutin, tryptophan, tocotrienol (alpha, beta, and gamma), zeaxanthin, gingko biloba, ginger, cat's claw (uncaria tomentosa), hypericum, aloe vera, evening primrose, garlic, capsicum, dong quai, ginseng, feverfew, fenugreek, echinacea, green tea, marshmallow, saw palmetto, tea tree oil, fish oil, payllium, kava-kava, licorice root, manonia aquifolium, hawthorne, hohimbr, tumeric, witch Hazel, valerian, mistletoe, bilberry, mushroom extract, astaxanthin, bee pollen, peppermint oil, beta-carotene, genistein, lutein, lycopene, the polyphenols or any combination thereof.

According to one embodiment, the topical formulations as provided herein may optionally include one or more suitable film forming agents. According to one embodiment, the one or more film forming agents may be present in an amount of from about 0.01% w/w to about 5.0% w/w based on the total weight of the final formulation. According to one embodiment, the one or more film forming agents may be present in an amount of from about 0.02% w/w to about 2.5% w/w. Suitable firm forming agents include, but are not limited to, liquid or solid emollient, surface active agents, gums, humectants, thickeners, powders, protein solutions or any combination thereof.

According to one embodiment, the topical formulations as provided herein may also include one or more emollients. According to one embodiment, the one or more film forming agents may be present in an amount of from about 0.1% w/w to about 10.0% w/w based on the total weight of the final formulation. According to one embodiment, the one or more film forming agents may be present in an amount of from about 2.0% w/w to about 7.5% w/w. Suitable emollients include, but are not limited to, mineral oil, fatty alcohols, alkyl esters, natural waxes (carnauba wax, bees wax, jojoba wax, etc.), silicones, silicone derivatives or any combination thereof.

According to one embodiment, the topical formulations as provided herein may also include one or more preservatives. According to one embodiment, the one or more preservatives is present in an amount of from about 0.01% w/w to about 5% w/w based on the total weight of the final formulation. According to one embodiment, the one or more preservatives is present in an amount of from about 0.02% w/w to about 2.5% w/w. Suitable preservatives include, but are not limited to, sodium benzoate, ascorbic acid, parabens, bronopol, methylisothiazolinone and their combinations. The anti-oxidant can be chosen, as an example, from amongst butylated hydroxytoluene (BHT), hydroxyanisole and tocopherol and their derivatives. The chelating agent can be chosen, for example, from the group formed by ethylenediaminetetraacetic acid (EDTA), ethylene glycol bis (2-aminoethyl ether) tetraacetic acid (EGTA) and citric acid or their salts.

According to one embodiment, the topical formulations as provided herein may also include one or more pH regulator systems. According to one embodiment, the one or more pH regulator systems is present in an amount of from about 0.01% w/w to about 5% w/w based on the total weight of the final formulation. According to one embodiment, the one or more pH regulator systems is present in an amount of from about 0.02 w/w to about 2.5% w/w. Suitable pH regulator systems can be chosen, as an example, from between a phosphate buffer solution, sodium hydroxide, citric acid, a citrate buffer solution, or any combination thereof.

According to one embodiment, the topical formulations as provided herein may optionally include one or more gelling agents. According to one embodiment, the one or more gelling agents is present in an amount of from about 0.01% w/w to about 5% w/w based on the total weight of the final formulation. According to one embodiment, the one or more gelling agents is present in an amount of from about 0.02% w/w to about 2.5% w/w. Suitable gelling agents include, but are not limited to, carbopols, poloxamers, carboxymethylcellulose (CMC), hydroxyethylcellulose (HEC), hydroxypropyl methylcellulose (HPMC), methylcellulose (MC) or any combination thereof.

According to one embodiment, the topical formulations as provided herein may optionally include one or more excipients. According to one embodiment, the one or more excipients may be present in an amount of from about 0.01% w/w to about 50% w/w based on the total weight of the final formulation. According to one embodiment, the one or more excipients may be present in an amount of from about 0.02% w/w to about 25% w/w. Suitable excipients include, but are not limited to, acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); alkalizing agents (ammonia solution, ammonium carbonate, diethanolamine, diisopropanolamine, potassium hydroxide, sodium bicarbonate, sodium borate, sodium carbonate, trolamine); antifoaming agents (dimethicone, simethicone); chelating agents; colorants (caramel, red, yellow, black or blends, ferric oxide); complexing agents, edetic acid, gentisic acid ethanolamide, oxyquinoline sulfate]; filtering aids (powdered cellulose, purified siliceous earth); humectants (glycerin, hexylene glycol, propylene glycol, sorbitol); polymers (e.g., cellulose acetate, alkyl celluloses, hydroxyalkyl, acrylic polymers and copolymers); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or viscosity-increasing agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum) or any combination thereof.

According to one embodiment, the topical formulations as provided herein may include one or more pharmacologically active agents or drugs suitable for administration according to the methods provided herein. Suitable pharmacologically active agents or drugs include all of the major therapeutic areas including, but not limited to, steroids (e.g., corticosteroids), anti-inflammatory agents, anti-infectives such as antibiotics and antiviral agents, analgesics and analgesic combinations, anorexics, antiarthritics, antiasthmatic agents, anticonvulsants, antihistamines, antiseptics, antimigraine preparations, antimotion sickness agents, antinauseants, diagnostics, hormones, hypnotics, immunosuppressives, muscle relaxants, parasympatholytics, psychostimulants, sedatives, tranquilizers, anesthetics, vitamins and combinations of the above. The amount of the pharmacologically active agent is an effective amount defined as a non-toxic but sufficient amount of a compound to provide the desired local or systemic effect and performance at a reasonable benefit/risk ratio attending any medical treatment.

According to one embodiment, the topical formulations provided herein exhibit a specific gravity at 25° C. of from about 0.50 to about 2.25. According to one embodiment, the topical formulations provided herein exhibit a specific gravity at 25° C. of from about 0.75 to about 1.75. According to one embodiment, the topical formulations provided herein exhibit a specific gravity at 25° C. of from about 1.00 to about 1.50.

According to one embodiment, the topical formulations provided herein exhibit a viscosity at 25° C. of from about 150000 cps to about 450000 cps. According to one embodiment, the topical formulations provided herein exhibit a viscosity at 25° C. of from about 200000 cps to about 400000 cps. According to one embodiment, the topical formulations provided herein exhibit a viscosity at 25° C. of from about 210000 cps to about 350000 cps.

Methods of preparing a topical formulation are provided. According to one embodiment, the method of preparing a topical formulation includes the step of preparing an aqueous phase. To prepare the aqueous phase, deionized water is added to an appropriate steam jacketed kettle with an agitator for scraping the walls of the kettle. According to one embodiment, the method of preparing the aqueous phase includes slowly heating the water to a temperature of from about 30° C. to about 55° C. with agitation. According to a particular embodiment, the water is slowly heated to a temperature of from about 40° C. to about 45° C. with agitation.

According to one embodiment, the step of preparing an aqueous phase includes the step of introducing at least one emulsion stabilizer/rheology modifier as provided herein (such as that commercially available as Carbopol® 934 cross-linked polyacrylic acid polymer) to the heated water and slowly heating the resulting composition to about 65° C. to about 90° C. with agitation. According to a particular embodiment, the emulsion stabilizer/rheology modifier is added to the water and heated slowly to about 75° C. to about 80° C. with agitation.

According to one embodiment, the step of preparing an aqueous phase includes the step of further introducing an emulsifier as provided herein to the heated water. Upon introduction, the resulting composition is maintained at about 55° C. to about 90° C. with agitation. According to a particular embodiment, the resulting composition is maintained at about 75° C. to about 80° C. with agitation. According to one embodiment, the emulsifier may be a polyethylene glycol sorbitan monostearate (such as that commercially available as Tween® 60 polyethlyene glycol sorbitan monostearate).

According to one embodiment, the method of preparing a topical formulation includes the step of preparing an oil phase. To prepare the oil phase, various components as provided herein are added to a separate vessel from that of the aqueous phase. Such components suitable for inclusion in the oil phase include, but are not limited to, any one or more thickening agent, emulsion stabilizer, emulsifier, rheology modifier, viscosity modifier, antioxidant, emollient, neutraceutical, vitamin, or any combination thereof. According to one embodiment, the components included in the oil phase include, but are not limited to, one or more of cetyl alcohol, beeswax, isopropyl palmitate, oleic acid (e.g., Emersol® 221), sorbitan stearate emulsifier (e.g., Arlacel™ 60), stearyl alcohol, stearic acid (e.g., Emersol® 132), glyceryl stearate (e.g., Emerest 2400), food shortening, at least one antioxidant such as BHT (butylated hydroxytoluene), vitamin E acetate, ascorbyl palmitate, at least one ester (e.g., Ceraphyl™ 424), or any combination thereof. According to one embodiment, the step of preparing an oil phase includes the step of mixing each of the individual oil phase components together in a single vessel. According to one embodiment, the step of preparing an oil phase includes the step of heating the various oil phase components provided herein. Any triglyceride or triglyceride-based components present in the oil phase may melt upon application of heat. According to one embodiment, the oil phase components may be heated to a temperature of from about 20° C. to about 80° C. According to a particular embodiment, the oil phase components may be heated to a temperature of from about 30° C. to about 70° C. the oil phase components may be heated to a temperature of from about 40° C. to about 60° C.

According to one embodiment, the method of preparing a topical formulation includes the step of forming an emulsion. According to one embodiment, the step of preparing an emulsion includes slowly adding the oil phase as provided herein to the aqueous phase as provided herein to form an emulsion. According to one embodiment, the step of preparing an emulsion further includes the step of mixing the aqueous phase and oil phase for about 5 minutes to about 40 minutes at a temperature of from about 60° C. to about 90° C. According to a particular embodiment, the step of preparing an emulsion further includes the step of mixing the aqueous phase and oil phase for about 10 minutes to about 20 minutes at a temperature of from about 70° C. to about 80° C.

According to one embodiment, the method of preparing a topical formulation includes the step of preparing a sodium hydroxide solution. The sodium hydroxide solution has a concentration of typically from about 1% w/v to about 20% w/v. According to one embodiment, the sodium hydroxide solution is about 10% w/v.

According to one embodiment, the method of preparing a topical formulation includes the step of slowly adding the sodium hydroxide solution to the emulsion. According to one embodiment, the resulting emulsion may then be heated from a temperature of from about 60° C. to about 90° C. for about 1 minute to about 20 minutes. According to a particular embodiment, the resulting emulsion may then be heated from a temperature of from about 70° C. to about 80° C. for about 5 minutes to about 15 minutes. According to one embodiment, the method includes the step of cooling the resulting emulsion to from about 30° C. to about 60° C. with slow agitation. According to a particular embodiment, the method includes the step of cooling the resulting emulsion to from about 40° C. to about 50° C. with slow agitation.

According to one embodiment, the method of preparing a topical formulation includes the step of weighing the one or more phospholipids included in the phospholipid component and adding the one or more phospholipids to a mixing vessel at ambient temperature. Any anhydrous phospholipids may be weighed in a dry hood or under nitrogen to prevent hydration and obtain accurate weight.

According to one embodiment, the method further includes the step of mixing the one or more phospholipids as provided herein which form the phospholipid component with one or more preservatives and, optionally, one or more carriers. According to a particular embodiment, the aforementioned method step includes the step of combining or mixing the phospholipid component as provided herein with disodium EDTA and, optionally, water. Accord to such an embodiment, the phospholipid component disperses into any water present upon mixing. According to one embodiment, the resulting mixture is agitated or mixed for about 5 minutes to about 40 minutes. According to a particular embodiment, the resulting mixture is agitated or mixed for about 10 minutes to about 20 minutes.

According to one embodiment, the method of preparing a topical formulation includes the step of adding the mixture to the emulsion at a temperature of from about 20° C. to about 50° C. to form an emulsion composition. According to a particular embodiment, the method of preparing a topical formulation includes the step of adding the mixture to the emulsion at a temperature of from about 30° C. to about 40° C.

According to one embodiment, the method of preparing a topical formulation includes the step of preparing a preservative/fragrance solution. To prepare the preservative/fragrance solution, one or more of an antioxidant, preservative, fragrance solution, or any combination thereof may be mixed together. According to one embodiment, the resulting preservative/fragrance solution is mixed for about 5 minutes to about 60 minutes. According to a particular embodiment, the resulting preservative/fragrance solution is mixed for about 10 minutes to about 40 minutes. According to a particular embodiment, the resulting solution is mixed for about 30 minutes. According to one embodiment, the preservative/fragrance solution is heated to about 25° C. to about 39° C. According to on embodiment, the preservative/fragrance solution is heated from about 30° C. to about 35° C.

According to one embodiment, the method of preparing a topical formulation includes the step of adding the preservative/fragrance solution to the emulsion composition. According to one embodiment, the resulting emulsion composition may then be slowly cooled from about 10° C. to about 45° C. while mixing. According to a particular embodiment, the resulting emulsion composition may then be slowly cooled from about 20° C. to about 35° C. while mixing.

According to one embodiment, the method of preparing a topical formulation includes the step of adding deionized water to the emulsion composition while mixing from about 10° C. to about 45° C. According to a particular embodiment, the method of preparing a topical formulation includes the step of adding deionized water to the emulsion composition while mixing from about 20° C. to about 35° C. According to one embodiment, the resulting emulsion composition is then left to stand for about 8 hours to about 16 hours.

According to one embodiment, the method of preparing a topical formulation includes the step of adding 0.1N sodium hydroxide or citric acid to the emulsion composition to adjust the pH to physiologic pH range. According to one embodiment, the physiologic pH range is form about 9.0 to about 5.0. According to a particular embodiment, the physiologic pH range is from about 7.5 to about 5.5. According to yet another embodiment, the physiologic pH range is form about 6.9 to about 6.2.

According to one embodiment, the method of preparing a topical formulation includes the step of homogenizing the emulsion composition at a temperature of from about 10° C. to about 45° C. to form a topical formulation. According to one embodiment, the method of preparing a topical formulation includes the step of homogenizing the emulsion composition at a temperature of from about 20° C. to about 35° C.

According to one embodiment, the topical formulations as provided herein may be applied to any part of the body where a skin application is acceptable. According to a particular embodiment, the topical formulations as provided herein encompass or may be made a part of a lotion, cream, ointment, foam, spray, emulsion, microemulsion, adhesive patch, oil, gel, a solid stick, salve, milk, paste or polish.

### EXAMPLE 1

A topical cream formulation was prepared. The topical cream formulation included the components (in the noted amounts) set forth below in Table 1.

To prepare the aqueous phase (Phase A—Table 1), deionized water was added to a steam jacketed kettle with an agitator for scraping the walls of the kettle. The water was slowly heated to a temperature of about 40° C. to about 45° C. with agitation. An emulsion stabilizer/rheology modifier (Carbopol® 934 cross-linked polyacrylic acid polymer) was added to the heated water and heated to about 75° C. to about 80° C. with agitation. An emulsifier (Tween® 60 polyethylene glycol sorbitan monostearate) was then added to the mixture and maintained at about 75° C. to about 80° C. with agitation.

To prepare the oil phase (Phase B—Table 2), cetyl alcohol, beeswax, isopropyl palmitate, oleic acid (e.g., Emersol® 221), sorbitan stearate emulsifier (e.g., Arlacel™ 60), stearyl alcohol, stearic acid (e.g., Emersol™ 132), glyceryl stearate (e.g., Emerest 2400), food shortening, BHT (butylated hydroxytoluene), and an ester (e.g., Ceraphyl™ 424) were introduced to a separate vessel from that of the aqueous phase.

An emulsion was formed by mixing the aqueous phase and oil phase and mixed for about 10 minutes to about 20 minutes at a temperature of from about 70° C. to about 80° C. A 10% sodium hydroxide solution (Phase C—Table 1) was then added to the emulsion and heated from a temperature of from about 70° C. to about 80° C. for about 5 minutes to about 15 minutes. The emulsion was then cooled down to about 40° C. to about 50° C. with slow agitation.

The phospholipid component was mixed with EDTA and further agitated or mixed for about 10 minutes to about 20 minutes (Phase D—Table 1). The resulting mixture was then added to and mixed with the emulsion at a temperature of from about 30° C. to about 40° C. to form an emulsion composition.

A preservative/fragrance solution was then prepared by mixing the components of Phase D of Table 1 for about 30 minutes and heating to about 30° C. to about 35° C. The preservative/fragrance solution was then added to the emulsion composition and slowly cooled from about 10° C. to about 45° C. while mixing.

Deionized water was then added to the emulsion composition while mixing at a temperature of from about 20° C. to about 35° C. The resulting emulsion composition was then left to stand for about 8 hours to about 16 hours. A 0.1N sodium hydroxide and citric acid were then added, as needed, to adjust the pH of the emulsion composition to a physiologic range. Finally, the emulsion was homogenized at a temperature of from about 20° C. to about 35° C. to form a cream.

TABLE 1

| | |
|---|---|
| Component | % w/w |

PHASE A

| | |
|---|---|
| water | 50.0 |
| Carbopol ® 934 (emulsion stabilizer/rheology | 0.3 |
| modifier) | |
| Tween ® 60 (Polyethylene glycol sorbitan | 1.8 |
| monostearate - emulsifier) | |

PHASE B

| | |
|---|---|
| Cetyl Alcohol | 1.0 |
| Beeswax | 0.4 |
| Isopropyl Palmitate | 5.0 |
| Emersol ® 221 (oleic acid) | 2.0 |
| Arlacel ™ 60 (sorbitan stearate emulsifier) | 1.2 |
| Stearyl Alcohol | 0.5 |
| Emersol ® 132 (Stearic acid) | 1.0 |
| Emerest 2400 (glyceryl stearate) | 1.0 |
| BBS (food shortening) | 10.0 |
| BHT (butylated hydroxytoluene - antioxidant) | 0.05 |
| Ceraphyl ™ 424 (esters for increasing body and | 1.0 |
| imparting waxy feel) | |

PHASE C

| | |
|---|---|
| NaOH 10% | 0.26 |

PHASE D

| | |
|---|---|
| Phospholipid Component | 5.0 |
| Disodium EDTA | 0.03 |

PHASE E

| | |
|---|---|
| Propylene Glycol | 3.0 |
| Parabens | 0.4 |
| Hedione (fragrance) | 0.02 |
| Benzyl Alcohol | 1.0 |
| Other Fragrance | 0.4 |

PHASE E

| | |
|---|---|
| water | Quantity |
| | sufficient to |
| | make 100 |

The anionic mole fraction of the total phospholipid component set forth in Table 1 included between from about 20 mole percent to about 100 mole percent of one or more net anionic phospholipid molecules as provided herein, with the balance of the remaining phospholipid component including one or more neutral zwitterionic phospholipids at physiologic pH values. Thus, the aggregate of the phospholipid component possessed a net negative charge at physiologic pH. The physiologic pH value was from about 9.0 to about 5.0.

### EXAMPLE 2

A topical cream formulation was prepared. The topical cream formulation included the components set forth below in Table 2. The cream was prepared utilizing the same steps as set forth in Example 1. The cream was found to have physical properties set forth in Table 3.

TABLE 2

| | |
|---|---|
| Component | % w/w |

PHASE A

| | |
|---|---|
| water | 60.0 |
| Carbopol ® 934 (emulsion stabilizer/rheology | 0.3 |
| modifier) | |
| Tween ® 60 (Polyethylene glycol sorbitan | 1.8 |
| monostearate - emulsifier) | |

PHASE B

| | |
|---|---|
| Cetyl Alcohol | 1.0 |
| Beeswax | 0.4 |
| Isopropyl Palmitate | 5.0 |
| Emersol ® 221 (oleic acid) | 4.0 |
| Arlacel ™ 60 (sorbitan stearate emulsifier) | 1.2 |
| Stearyl Alcohol | 0.5 |
| Emersol ® 132 (Stearic acid) | 1.0 |
| Emerest 2400 (glyceryl stearate) | 1.0 |
| BBS (food shortening) | 10.0 |
| BHT (butylated hydroxytoluene - antioxidant) | 0.05 |
| Vitamin E Acetate | 0.025 |
| Ascorbyl Palmitate | 0.10 |
| Ceraphyl ™ 424 (esters for increasing body and | 1.0 |
| imparting waxy feel) | |

PHASE C

| | |
|---|---|
| NaOH 10% | 1.3 |

PHASE D

| | |
|---|---|
| Phospholipid Component | 1.0 |
| Water | 2.5 |
| Disodium EDTA | 0.03 |

PHASE E

| | |
|---|---|
| Propylene Glycol | 3.0 |
| Parabens | 0.15 |
| Benzyl Alcohol | 1.0 |
| Fragrance | 0.3 |

PHASE E

| | |
|---|---|
| water | Quantity |
| | sufficient |
| | to make |
| | 100 |

The anionic mole fraction of the total phospholipid component set forth in Table 2 included between from about 20 mole percent to about 100 mole percent of one or more net anionic phospholipid molecules, with the balance of the remaining phospholipid component including one or more neutral zwitterionic phospholipids at physiologic pH values. The physiologic pH value was from about 9.0 to about 5.0.

TABLE 3

| | |
|---|---|
| Appearance | Viscous smooth cream |
| Color | White, colorless |
| pH 10% slurry | 6.2-7.0 |
| Specific gravity @ 25° C. | 1.026-1.25 |
| Viscosity @ 25° C. | 224000 Cps-336000 Cps |

All publications, patents and patent applications cited in this specification are incorporated herein by reference for the teaching to which such citation is used.

The specific responses observed may vary according to and depending on the particular active compound selected or whether there are present carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A method of preparing a topical phospholipid formulation comprising the steps of:
preparing an aqueous phase;
preparing an oil phase;
mixing the oil phase and aqueous phase to form an emulsion;
introducing at least one mixture comprising a phospholipid component to the emulsion to form an emulsion composition;
adjusting the pH of the emulsion composition to physiologic pH; and
homogenizing the emulsion composition to form the topical phospholipid formulation,
wherein the phospholipid component includes an anionic mole fraction of between from about 20 mole percent to about 100 mole percent of one or more net anionic phospholipid molecules, with the remaining balance of the phospholipid component including one or more neutral zwitterionic phospholipids at the physiologic pH.

2. The method of claim 2, wherein the physiologic pH is from about 9.0 to about 5.0.

3. The method of claim 1, wherein the step of preparing the aqueous phase includes the steps of:
heating water; and
adding one or more of an emulsifier, emulsion stabilizer, and rheology modifier to the water.

4. The method of claim 1, wherein the step of preparing an oil phase includes mixing at least two or more components selected from the group consisting of cetyl alcohol, beeswax, isopropyl palmitate, oleic acid, sorbitan stearate, stearyl alcohol, stearic acid, glyceryl stearate, food shortening, antioxidant, vitamin E acetate, ascorbyl palmitate, and an ester.

5. The method of claim 1, wherein the step of mixing the oil phase and aqueous phase to form an emulsion includes mixing for about 10 minutes to about 20 minutes at a temperature of from about 70° C. to about 80° C.

6. The method of claim 1, wherein the step of adjusting the pH is carried out by introducing 0.1N sodium hydroxide or citric acid to the emulsion.

7. The method of claim 1, wherein the step of homogenizing the emulsion composition is carried out at from about 20° C. to about 35° C.

8. The method of claim 1, further comprising the step of introducing a 10% w/v sodium hydroxide solution to the emulsion.

9. The method of claim 1, further comprising the step of introducing one or more preservative/fragrance solution to the emulsion composition.

10. The method of claim 1, further comprising the step of combining the phospholipid component with one or more preservative and, optionally, one or more carrier, to form the mixture.

11. The method of claim 10, wherein the carrier is water, an alcohol, or a combination thereof.

12. The method of claim 1, wherein the phospholipid component is present in a concentration of from about 0.1% w/w to about 10% w/w based on the total weight of the formulation.

13. The method of claim 1, wherein the phospholipid component comprises one or more phospholipid molecules selected from the group consisting of lysophosphatidic acid, lysodiphosphatidylglycerol, phosphatidylglycerol, lysophosphatidylethanolamine, phosphatidic acid, diphosphatidylglycerol, ethanolamine plasmalogen, phosphatidylethanolamine, phosphatidylserine, dimethylphosphatidylethanolamine, lysophosphatidylcholine, phosphatidylinositol, and phosphatidylcholine.

14. The method of claim 1, wherein the phospholipid component includes an anionic mole fraction of between from about 20 mole percent to about 40 mole percent of one or more net anionic phospholipid molecules.

15. The method of claim 1, wherein the topical phospholipid formulation is a lotion, hydrogel, oil, emulsion, paste, polish, or cream.
